(19) United States
(12) Patent Application Publication
(10) Pub. No.: US 2010/0056895 A1
(43) Pub. Date: Mar. 4, 2010

(54) Indicia-coded medical diagnostic apparatus

(76) Inventors: Mary Darlene Temple, Imperial, PA (US); Bradley Allen Temple, Imperial, PA (US)

Correspondence Address: Mary Darlene Temple, 8325 Hilltop Circle Dr., Imperial, PA, 15126, US

(21) Appl. No.: 12/590,226

(22) Filed: Nov. 3, 2009

Related U.S. Application Data
(62) Division of application No. 10/725,806, filed on Dec. 2, 2003.

(51) Int. Cl. A61B 5/00; G08B 6/00

(52) U.S. Cl. 600/365; 340/407.2

(57) ABSTRACT

An apparatus for quantitative measurement of glucose or other biological substance. analysis. The apparatus accepts a sample of blood or biological substance onto or into a diagnostic reagent test device which has a reagent carrier and mode of relaying to the instrument the identifying reagent characteristics. The instrument includes a housing structure having an indicia-coded display and multi-function keypad, a programmable microcontroller which enables the apparatus to emanate or emit said indicia display or a plurality of indicia displays corresponding to an arrangement of quantitative blood glucose or biological substance values wherein said values are programmed into said apparatus and are determinate upon known quantitative analysis ranges of glucose or other analyzed components in sampled blood or biological substances.

### CROSS REFERENCES TO RELATED APPLICATIONS

The present invention is a divisional application of pending U.S. patent application Ser. No. 10/725,806 filed on Dec. 2, 2003 titled “Apparatus and Method for Delivery of Medication”.

### FEDERALLY SPONSORED RESEARCH OR DEVELOPMENT

Not Applicable

### REFERENCE TO SEQUENCE LISTING

Not Applicable

### BACKGROUND OF THE INVENTION

Diabetes is a chronic, complex metabolic disease that results in the inability of the body to properly maintain and use carbohydrates, fats, and proteins. It results from the interaction of various hereditary and environmental factors and is characterized by high blood glucose levels caused by a deficiency in insulin production or an impairment of its utilization.

The risk of complications occurring with diabetes can be greatly reduced if the patient maintains good control of his/her blood glucose levels. Reduction of blood glucose concentrations in the blood of patients with diabetes mellitus prevents the development of diabetic complications. Good control of blood glucose levels can be defined as having an adequate amount of insulin to utilize the amount of glucose in the body. Currently, adequate blood glucose control is difficult to achieve in many patients with diabetes due to their inability to visually or cognitively perform the functions necessary to achieve optimum results; for instance, viewing the blood glucose result displayed by the apparatus used to measure their blood glucose levels and/or preparing and delivering the proper dosage of medication for the glucose level displayed by their monitoring device. In order to achieve optimum levels of blood glucose control in real time, the correct amount of insulin to blood glucose level must be achieved. Patients with cognitive or physical limitations are routinely prescribed a constant dose of insulin at varying intervals throughout the day in the hope that it will keep their blood glucose levels in good control. Often, the constant insulin dose is prescribed at a much lower dose than what is actually needed due to not desiring an adverse insulin reaction. Patients are given this prescribed low dosage simply because they cannot navigate the process of reading the blood glucose result displayed on their glucometer and preparing the actual correct dosage of insulin needed for their blood glucose level. As a result, optimum blood glucose control is not achieved. Examples of patients who are most at risk for having the aforementioned physical or cognitive challenges resulting in complications due to poor blood glucose control are, but not limited to, the elderly, the very young, the visually or cognitively impaired and handicapped diabetics. The present invention seeks to remedy the challenges in a very simplistic way.

A preferred embodiment of the invention is the matching of indicia to obtain optimum blood glucose control. This is achieved by matching the indicia emanated/emitted from the apparatus to the indicia of a plurality of matching indicia-coded and pre-filled syringes, enabling the diabetic to navigate the process of insulin administration easily. When the glucose level falls within a pre-programmed numerical range, visual and/or tactile indicia corresponding to said range will be displayed on the screen or emitted from the device. In cases of life-threatening blood glucose or biological substance analysis, the device has the capability of emitting tactile and visual indicia to the user as well as sending the life-threatening results to pre-determined recipient such as a caregiver or physician's office via wireless communication. When combined with the medication delivery system of patent application Ser. No. 10/725,806, the parent application of this invention, it provides a failsafe method for physically or cognitively challenged patients to administer the correct dosage of medication

In another embodiment of the present invention, pre-filled and indicia-coded syringes corresponding to the aforementioned apparatus display indicia are described. Insulin is commonly delivered via syringes in specified doses and administered during different intervals of time throughout the day. The current standard insulin syringe is clear, small, and utilizes incremental dosage markings that are very minute and difficult to read. Current remedies involve the enhancement of said markings, see for example, U.S. Pat. No. 72,0381 (1903), for Hypodermic Syringe. In patients with moderate to severe visual impairment the magnification or enhancement processes currently in use is often not an adequate solution to ensuring correct dosing. By enhancing the syringes with indicia corresponding to the glucose monitor display, and having a caregiver/medical professional pre-fill the syringes with medication corresponding to the correct blood glucose range displayed by the glucose monitor display, a failsafe method of delivering adequate doses of insulin for the blood glucose level is achieved.

Prior art blood glucose monitoring devices relate to a quantitative measurement of bodily fluid produced by reactive agent. The present invention incorporates the known technology of these devices but differs in that the device incorporates a programmable microcontroller for programming the numerical ranges of diagnostic test analysis into the device. These numerical ranges are then matched to indicia or multiple indicia through the microcontroller whereby the indicia is emanated on the screen of the apparatus or emitted from the device. The user then selects the appropriate dosage of medication that matches the emanated or emitted indicia In the case of diabetics and insulin administration, the user would select a pre-filled syringe with matching indicia corresponding to the indicia of the apparatus display.

The prior art also involves a reagent chemical reaction with respect to the blood glucose and changing color, and a blood glucose meter for reading the change in color indicating the blood glucose level. The level of blood glucose, when measured by a glucometer, is read from a unit carrying the blood chemistry through the well known process of reflectometers based on the principle of glucose oxidation. The present invention uses the same principles of reading blood glucose or bodily fluid samples known to those skilled in the art but differs in the way the results are displayed, recorded and transmitted. My apparatus is programmed by the user/caregiver/medical professional with regard to blood glucose or bodily fluid ranges and these ranges are then converted via microprocessor to emanate or emit an indicia coded display or tactile emission based on the programmed value ranges.

Another object of the present invention is an electromechanical assembly which contains the microprocessor including the software, mechanical and electromechanical apparatus, batteries, and related circuitry that causes the electrical and electromechanical functional operation of the apparatus.

Still, another object of the present invention is a medical diagnostic system which is software controlled and software intelligent. The system is programmed very simply by an operator and controlled by commands programmed into the software.

A need therefore exists for an improved medication diagnostic apparatus comprising indicia.

### BRIEF SUMMARY OF THE INVENTION

The present invention pertains to a medical apparatus, and more particularly, to a glucose medical monitoring diagnostic device for sampling and analyzing blood or any biological substance or substances for specific readings as to qualities of the blood or biological substance.

One specific use of the present invention is for measuring the accumulation of blood glucose for diabetics, correlating the display to a plurality of diagnostic ranges corresponding to indicia, such as those found in prescribed sliding scales of insulin to quantitative blood sugar levels, and further correlating those ranges, via microprocessors, to illuminate/emanate indicia to be displayed by this apparatus. The diagnostic apparatus is a programmable, battery-operated, portable, diagnostic unit, which through known processes from prior art, comprises a means for accepting a sample of blood or biological substance, a means for analyzing through quantitative measurement said sample and a means for displaying the diagnostic result. The present invention comprises a means for the user/caregiver/medical professional to program a pre-determined set of quantitative analysis ranges corresponding to indicia and a means to emanate/emit the programmed indicia on the apparatus display. The apparatus has an optional means for time stamping, dating, recording and printing said analysis. The apparatus further comprises a means for transmission of the results to a caregiver or medical professional and a means to alert the user as well as the recipient of a transmission to abnormal results. The device is programmed by the user, medical professional or caregiver and provides a means for easy recognition of blood glucose or biological substance analysis and administration of medication corresponding to said analysis in patients/users with physical or cognitive impairment.

For a better understanding of the present invention, together with other and further features and advantages thereof, reference is made to the following description, taken in conjunction with the accompanying drawings, and the scope of the invention that will be pointed out in the appended claims.

### BRIEF DESCRIPTION OF THE DRAWINGS

FIG. 1 illustrates a perspective front closed view of the apparatus.

FIG. 2 illustrates a perspective closed back view of the apparatus with open battery compartment.

FIG. 2A illustrates a sectional view of the battery compartment cover.

FIG. 3 illustrates a perspective side view of the apparatus

FIG. 4 illustrates a perspective bottom view of the apparatus.

FIG. 5 illustrates an open front view of the apparatus with programmable keypad

FIG. 6 illustrates a sectional view of the programmable keypad.

### DETAILED DESCRIPTION OF THE INVENTION

The preferred embodiment of the present invention will now be described. In the preferred embodiment, there is described indicia-distinctive components that, when combined with a system of use, enable insulin dependent diabetics a safe and easy way to control their blood glucose levels. It will be appreciated that the present invention is useful for diabetics who would benefit from the use of a sliding scale of insulin to control their diabetes. Though the invention may be described in connection with insulin, it may be used for any medication regimen.

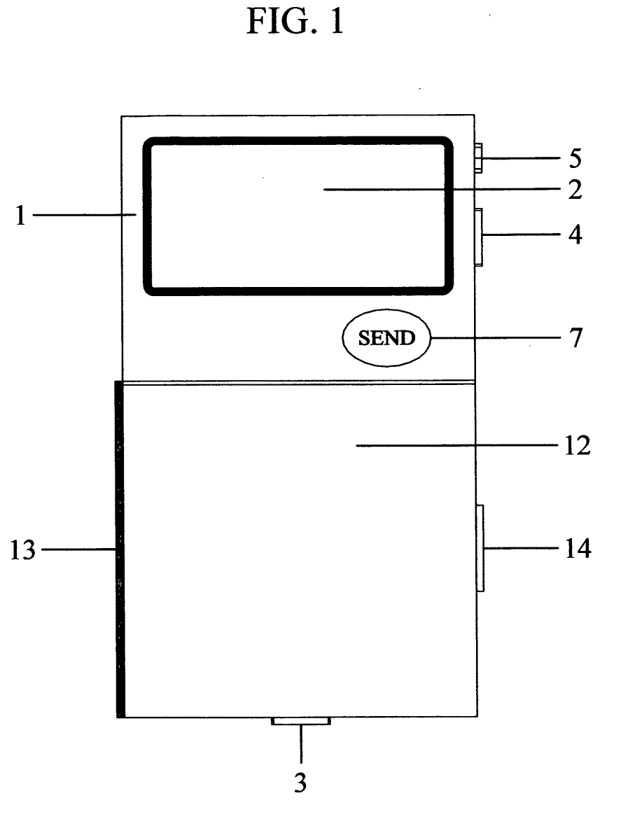

Turning now to the drawings, FIG. 1 illustrates a front closed view of the apparatus. This view represents the apparatus as it would appear to the user/patient. The apparatus as seen in this view incorporates a front housing 1 panel with an inlet 3 for inserting a blood glucose or reagent test strip for analysis. The present invention incorporates two sources of power. One source is plug-in power port 5 seen in this illustration for AC to DC current. The second source is a battery which will be further described in subsequent views. An external data port 4 is illustrated as a means for transmitting stored test results directly to a computer database. A “Send” button 7 is incorporated as a means for the user to send wireless transmissions of test results to medical professionals and/or caregivers. The present invention also has the capability sending automatic wireless alerts to predetermined caregivers and/or medical professionals when abnormal test results are detected by the apparatus. This capability is internally controlled through a microcontroller with technology known to those skilled in the process. The apparatus as seen in this view illustrates a closed hinged 13 programmable keypad cover 12 with latch 14 incorporated to prevent inadvertent programming of the keypad which is illustrated in subsequent views. The test strip is analyzed by methods known to those skilled in the art. The result and corresponding indicia for the range the result falls within are displayed by the apparatus on a large illuminated LCD display 2. The displayed indicia corresponds to a pre-determined blood glucose or biological sample range that is programmed into the apparatus via a keypad 9 shown on further views and generated through a microcontroller. The user/patient matches the emanated/emitted indicia displayed by the apparatus to the indicia corresponding to the same range indicated by the indicia of a pre-filled and indicia-coded syringe.

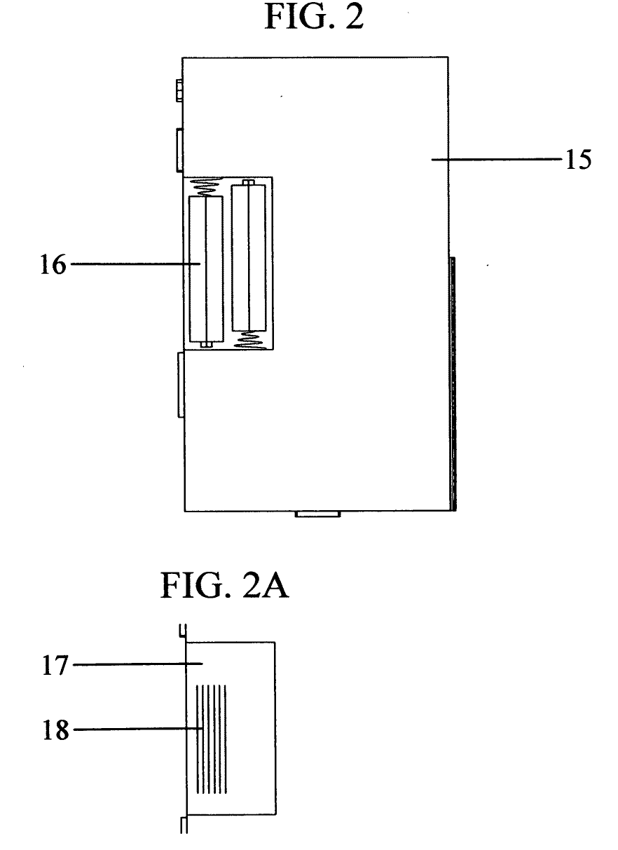

FIG. 2 illustrates a closed back perspective view of the apparatus. A back housing 15 connects to the front housing 1 as a means to keep contaminates from penetrating the apparatus. The alternate power source for the apparatus is a battery which is contained within a battery compartment 16 as seen in this view.

FIG. 2A illustrates a battery compartment cover 17 with finger grip 18.

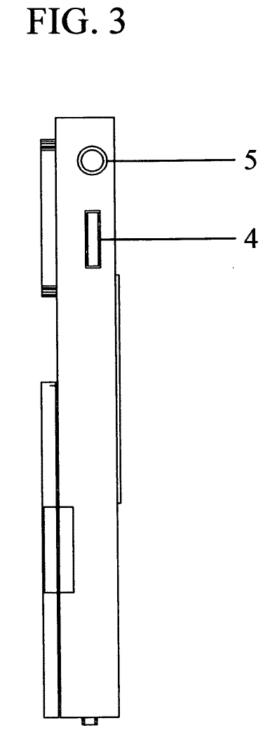

FIG. 3 illustrates a side view of the apparatus with plug-in 1 power port 5 and external data port 4 shown.

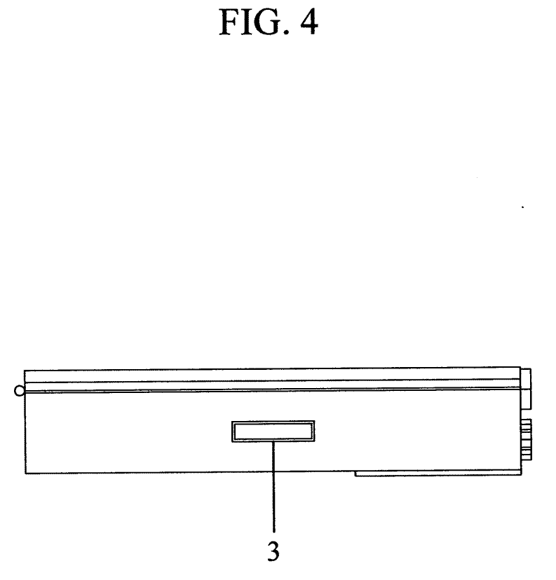

FIG. 4 illustrates a preferred embodiment of the invention with a bottom view of the apparatus with test strip inlet 3 incorporated.

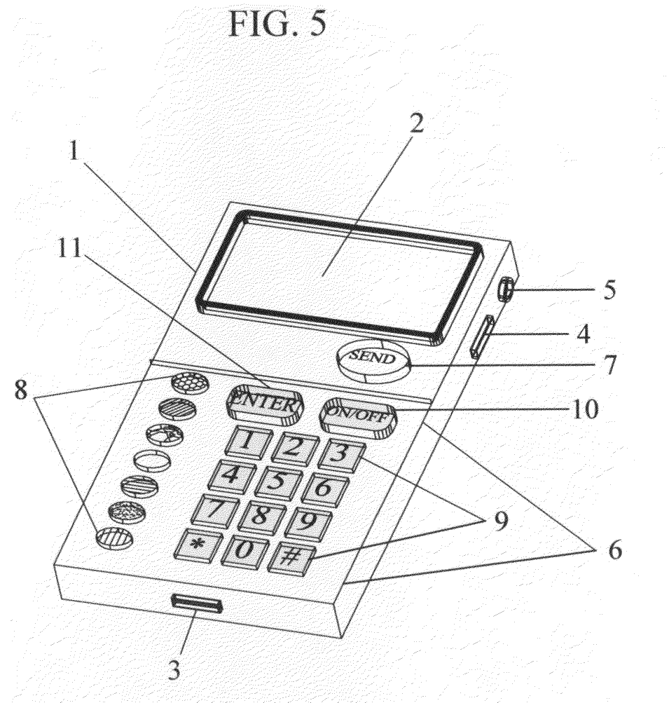

FIG. 5 illustrates a perspective open view of the apparatus with the programming keypad 6 uncovered. This would be the view as seen by the medical professional/caregiver/user programming the apparatus. An LCD display 2 is illustrated and functions as the preferred embodiment of the invention whereby the user matches emanated and/or emitted indicia from the display to the aforementioned indicia of pre-filled syringes of parent application Ser. No. 10/725,806.

A power button 10 allows the user/caregiver/programmer to initialize the programming process. Indicia-coded programming keys 8 allow the programmer to select the indicia to be emanated/emitted from the apparatus. Numerical programming keys 9 allow the programmer to enter numerical value ranges corresponding to the selected indicia-coded keys 8. The “Enter” key 11 is incorporated for use with prompts generated via the microcontroller and displayed on the LCD display 2.

In use the patient inserts a test strip into the apparatus via the test strip inlet 3. This action activates the apparatus. The apparatus analyzes the test strip by methods known to those skilled in the art and subsequently, through programming of the microcontroller, emits/emanates indicia via the LCD display 2. The microcontroller is programmed by the user/caregiver/medical professional via the programmable keypad 6 and indicia keys 8 to display indicia corresponding to a quantitative value range which is again pre-programmed by the user to correspond to the indicia in which the analyzed result falls within.

In use, the programmer/caregiver/medical professional opens the hinged cover, 13 and 14 respectively. The programmer then presses the On/Off button 10 to activate the programming sequence. The LCD display 2 prompts the programmer to “Enter Pin”. A pre-determined pin number is then programmed using the numerical keys 9. The LCD display 2 prompts the programmer to, “Select Indicia” (color, number, letters,) Programmer selects an indicia key 8 from those located at the left of the programming keypad The indicia selected by the programmer illuminates on the LCD display 2. While still illuminated with the selected indicia, the LCD display 2 prompts the programmer to “Enter Low Range”. Programmer enters low range for the selected indicia from the numerical keys 9. While continuing to illuminate with the selected indicia, the LCD display 2 prompts the programmer to “Enter High Range.” Programmer enters high range for the selected indicia on the numerical keypad 9. When complete the LCD display 2 illuminates with the selected indicia as well as the low and high ranges for the selected indicia. The LCD display 2 then prompts the programmer to press “Enter” 11 on the keypad if correct. Programmer presses “Enter” 11 if correct. LCD display 2 prompts programmer to select next indicia. The programmer repeats the process of entering all numerical ranges for each indicia 8 on the numerical keypad 9. When all indicia have been selected the LCD display 2 will prompt, “Programming Complete? If yes, press Enter” 11.

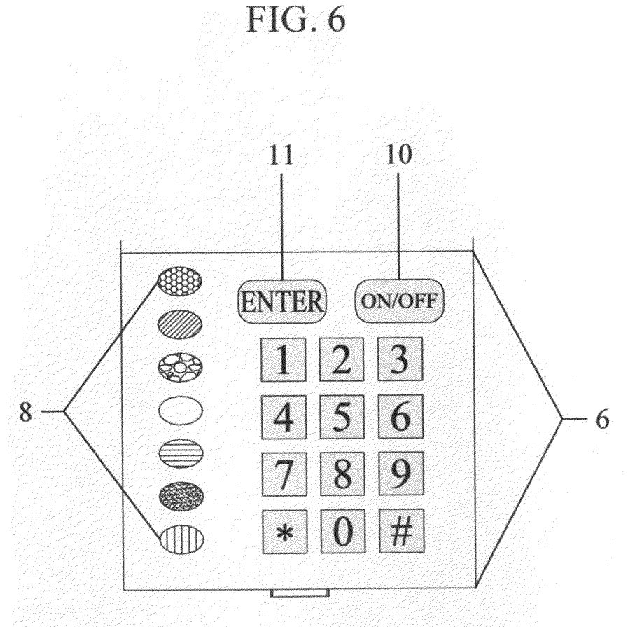

FIG. 6 illustrates a detailed view of the programmable keypad. An “On/Off” button 10 is described as the initiating programmable keypad function. Indicia selection keys 8 as well as numerical keys 9 are illustrated as a means for entering programming data. An enter key 11 serves as a means for programming progression.

If not otherwise stated herein, it is to be assumed that all patents, patent applications, patent publications and other publications (including web-based publications) mentioned and cited herein are hereby fully incorporated by reference herein as if set forth in their entirety.

Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

1. In a portable, hand-held apparatus for quantitative measurement of blood glucose or other biological substances and of the type comprising an arrangement for accepting a sample of a patient's blood or other biological substance, a means for analyzing said blood or other biological substance, further comprising a reagent test device which has a reagent carrier and means of relaying to the apparatus the identifying reagent characteristics and an arrangement for displaying said quantitative measurement in which the improvement comprises;
a. a means for programming a pre-determined blood glucose or biological fluid analysis range, or plurality of ranges into said apparatus, whereby said analysis range or ranges correspond to pre-determined quantitative measurement values of blood glucose or other biological substance values, further comprising;
b. a means for displaying or emitting said analysis range or plurality of ranges, comprising indicia.

2. The apparatus of claim 1, wherein said indicia are color based.

3. The apparatus of claim 1, wherein said indicia are tactile based.

4. The apparatus of claim 1 wherein said indicia is selected from a group consisting of colors, numbers, letters, graphic symbols, tactile symbols and machine-readable bar codes or a combination thereof.

5. The apparatus of claim 1 wherein said indicia further correspond to the indicia of an indicia-coded medication dispensing device or a plurality of said devices.

6. The apparatus of claim 1 wherein said indicia further correspond to an indicia-coded medication dispensing device or plurality of said devices wherein said device or devices comprise pre-filled syringes containing a pre-determined dose or dosages of medication.

7. The apparatus of claim 1 wherein said indicia further correspond to indicia or plurality of said indicia of a medication chart whereon a pre-determined dose or dosages of medication are transcribed.

8. The apparatus of claim 1, further comprising a means for recording said quantitative measurement of blood glucose or other biological substance analysis.

9. The apparatus of claim 1, further comprising a means for printing said quantitative measurement of blood glucose or other biological substance analysis whereon said printing further comprises;
a. a time and date stamp and,
b. removable adhesive labels

10. The apparatus of claim 1, further comprising a means for transmission of said quantitative measurement of blood glucose or other biological substance analysis whereby said analysis can be retrieved by a caregiver or medical professional.

11. The apparatus of claim 1, further comprising a means for alerting said user to abnormal quantitative measurement analysis wherein said alert is in the form of said indicia or a plurality of said indicia.

12. The apparatus of claim 1 further comprising a means for alerting said transmission recipient to abnormal quantitative measurement analysis.

13. A housing comprising a rectangular shape and LCD display, further comprising a programmable keypad with numerical and function keys whereon said value range or plurality of ranges are programmed.

14. The housing of claim 12 further comprising a hinged cover to protect said keypad from inadvertent programming.

15. The housing of claim 12 further comprising a data port wherein said quantitative measurement analysis is transmitted.
